(19) United States
(12) Patent Application Publication
(10) Pub. No.: US 2020/0338134 A1
(43) Pub. Date: Oct. 29, 2020

(54) MUSCLE TREATMENT METHOD USING MYOD MUTANT

(71) Applicant: Cleveland State University, Cleveland, OH (US)

(72) Inventor: Crystal M. Weyman, Willoughby Hills, OH (US)

(73) Assignee: Cleveland State University, Cleveland, OH (US)

(21) Appl. No.: 16/858,762

(22) Filed: Apr. 27, 2020

Related U.S. Application Data
(60) Provisional application No. 62/838,977, filed on Apr. 26, 2019.

(51) Int. Cl. A61K 35/34; A61K 9/00; C12N 15/85

(52) U.S. Cl. CPC A61K 35/34; C12N 2800/10; C12N 15/85; A61K 9/0019

(57) ABSTRACT

A method for treating a patient with a muscle condition includes transplanting multipotent cells into the patient. A MyoD mutant that has been modified (e.g., to decrease PUMA expression relative to myogenin expression) has been transferred into the multipotent cells. The modification may lead to decreased apoptosis relative to differentiation. The transplantation may be local (i.e., directly to the muscle).

The present application claims the priority benefit of U.S. Provisional Patent Application Ser. No. 62/838,977, Apr. 26, 2019, the disclosure of which is incorporated herein by reference.

### BACKGROUND

Skeletal muscle represents 40-45% of the average total human body mass. Musculoskeletal disorders are the primary cause of disability in the U.S. resulting in a financial burden of $800 billion annually. There are 4.5 million cases annually of volumetric muscle loss. Volumetric muscle loss is a skeletal muscle injury with chronic functional impairment which may be caused by traffic accidents, blast/combat trauma (80% of injuries), surgical trauma, and sports injuries (35-55%). Duchenne muscular dystrophy (DMD) results from a loss of functional dystrophin (structural protein) and effects about 30 out of 100,000 males. It is typically fatal in the 20s-40s and qualifies for orphan drug designation by the Food and Drug Administration (FDA).

Glucocorticoids (e.g., prednisone and deflazacort) are the current standard of care and have been used for over 20 years to treat DMD but only slow the progression of the disease. Additionally, they have adverse side effects such as weight gain, behavioral changes, immune suppression, and hirsuitism. Eteplirsen is another medication but it is specific to 13% of about 15,000 DMD patients specifically amenable to exon 51 skipping.

Cell therapy treatments have been attempted but have been largely unsuccessful due to the loss of transplanted donor cells. In other words, muscle stem cells or modified stem cells tend to die via apoptosis (programmed cell death) prior to fusion with the patient's pre-existing muscle tissue.

Other treatments include surgery (autologous muscle transfer), biological scaffolding, and physical therapy. However, there are currently no curative treatments for muscle wasting, injury and disease.

It is desirable to develop new systems and methods for treating muscles, particular for muscle wasting, damage and dystrophy.

### BRIEF DESCRIPTION

The present disclosure relates to systems and methods for treating muscles.

Disclosed, in some embodiments, is a method for treating a patient with a muscle condition including: transplanting multipotent cells into muscle tissue. A MyoD mutant has been transferred into the multipotent cells. The MyoD mutant may have been modified to decrease PUMA expression relative to myogenin expression.

In some embodiments, the MyoD mutant is MyoD s200a.

The MyoD mutant may have been modified to replace from 1 to about 8 amino acids.

In some embodiments, the MyoD mutant has been modified to replace at least one instance of serine with alanine.

The multipotent cells may be transplanted via local injection into the muscle tissue.

In some embodiments, the muscle condition is muscular dystrophy.

The muscular dystrophy may be Duchenne muscular dystrophy, Becker muscular dystrophy, myotonic muscular dystrophy, limb-girdle muscular dystrophy, facioscapulohumeral muscular dystrophy, congenital muscular dystrophy, oculopharyngeal muscular dystrophy, distal muscular dystrophy, or Emery-Dreifuss muscular dystrophy.

In some embodiments, the muscle condition is an acute muscle injury.

The muscle condition may be muscle wasting.

In some embodiments, the muscle condition is sarcopenia.

The MyoD mutant may be characterized by all serines and threonines located next to proline having been mutated to alanine.

Disclosed, in other embodiments, is a method for treating a patient with a muscle condition. The method includes forming a MyoD mutant, transferring the MyoD mutant into multipotent stem cells, and transferring the multipotent stem cells into muscle tissue of the patient.

The transferring of the MyoD mutant into the multipotent stem cells may be performed via transfection.

In some embodiments, the transfection is performed using a plasmid.

pCS2 plasmids represent a non-limiting embodiment of plasmids that may be used in the transfection.

These and other non-limiting characteristics are more particularly described below and in the appended materials.

### BRIEF DESCRIPTION OF THE DRAWINGS

FIG. 1 is a flow chart illustrating a method for treating muscle tissue in accordance with some embodiments of the present disclosure.

FIG. 2 schematically illustrates a muscle.

FIG. 3 includes graphs for PUMA expression of 10T1/2 fibroblasts after transfection with MyoD s200a mutant.

FIG. 4 includes graphs for myogenin expression of 10T1/2 fibroblasts after transfection with MyoD s200a mutant.

### DETAILED DESCRIPTION

The present disclosure may be understood more readily by reference to the following detailed description of desired embodiments included therein and the appended materials. In the following specification and the claims which follow, reference will be made to a number of terms which shall be defined to have the following meanings.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent can be used in practice or testing of the present disclosure. All publications, patent applications, patents, and other references mentioned herein are incorporated by reference in their entirety. The materials, methods, and articles disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases that require the presence of the named ingredients/steps and permit the presence of other ingredients/steps. However, such description should be construed as also describing compositions, mixtures, or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values in the specification should be understood to include numerical values which are the same when reduced to the same number of significant figures and numerical values which differ from the stated value by less than the experimental error of the conventional measurement technique of the type used to determine the particular value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The present disclosure relates to methods, systems, and compositions for treating a muscle condition. The methods generally include transplanting multipotent cells into muscle tissue after a MyoD mutant has been transferred into the multipotent cells. In some embodiments, the multipotent cells have been transfected with the MyoD mutant. The MyoD mutant may have been modified to reduce or eliminate programmed cell death (apoptosis).

Apoptosis is induced at the same time as specialization (differentiation) in skeletal muscle and other tissues. In skeletal muscle stem cells (myoblasts), these two processes are both initiated by differentiation cues. The transcription factor MyoD, long known at the pioneering molecule responsible for differentiation, is also controlling apoptosis.

The treatments of the present disclosure use mutants of MyoD that allow for differentiation but not apoptosis. By reducing or eliminating apoptosis of stem cells, the efficiency of stem cell treatments can be increased because fewer stem cells will die prior to fusion with pre-existing muscles.

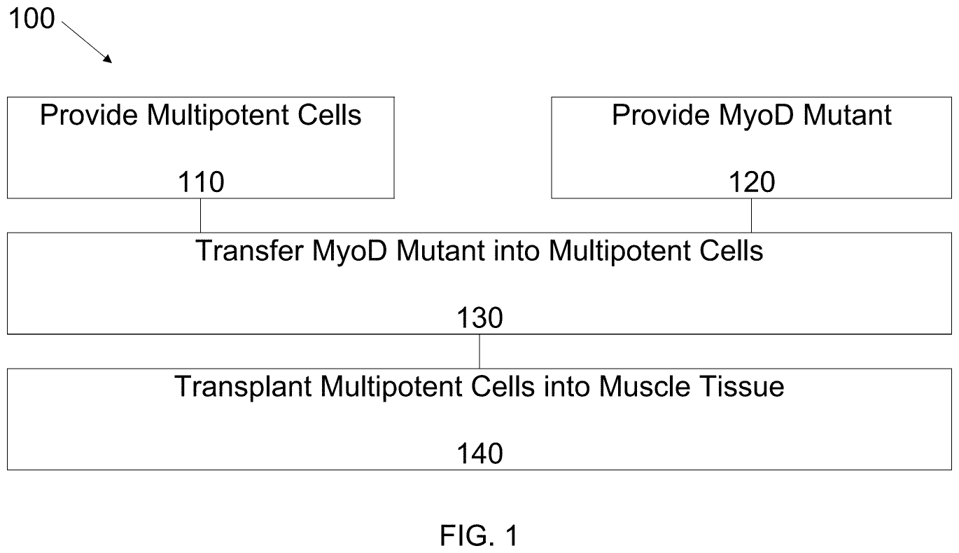

FIG. 1 illustrates a non-limiting method 100 for treating a muscle condition in accordance with some embodiments of the present disclosure. The method 100 includes providing multipotent cells 110, providing a MyoD mutant 120, transferring the MyoD mutant into the multipotent cells 130, and transplanting the multipotent cells into muscle tissue 140.

The method 100 may be used to treat muscular dystrophy, muscle wasting, and/or a muscle injury.

The muscular dystrophy may be Duchenne muscular dystrophy or Becker muscular dystrophy. In other embodiments, the muscular dystrophy is myotonic, limb-girdle, facioscapulohumeral, congenital, oculopharyngeal, distal, or Emery-Dreifuss muscular dystrophy.

The muscle wasting may be amyotrophic lateral sclerosis (ALS).

The muscle injury may be an acute injury or a chronic injury.

In some embodiments, the method is used to treat atrophy or sarcopenia.

The provided 110 multipotent cells may be obtained from a donor and/or autologously (i.e., from the patient being treated). The multipotent cells may be stem cells or modified stem cells.

In some embodiments, the multipotent cells are fibroblasts.

A MyoD mutant is provided 120. As used herein, the term “MyoD mutant” refers to MyoD which has been modified. In some embodiments, the modification decreases p53 upregulated modulator of apoptosis (PUMA) expression relative to myogenin expression. The modification leads to decreased apoptosis relative to differentiation. In some embodiments, apoptosis is reduced or eliminated while still allowing differentiation. Accordingly, the stem cells survive long enough to differentiate and fuse with the existing muscle tissue of a patient being treated.

The MyoD mutant may be modified by substituting one or more amino acids in MyoD. In some embodiments, the modification includes substituting from 1 to about 8 amino acids, including from 1 to about 7 and from 2 to about 5 amino acids. The modification may include replacing at least one instance of serine and/or threonine with alanine. The same kinase enzyme may be used to phosphorylate the hydroxyl group of these amino acids.

In particular embodiments, the MyoD mutant is MyoD s200a. MyoD s200a is a MyoD mutant wherein serine is replaced with alanine at the 200 position of MyoD. The substitution may prevent phosphorylation of the MyoD mutant.

The MyoD mutant is incorporated (e.g., transferred or transfected) 130 into the multipotent stem cells. Transfection 130 may be performed using pCS2 plasmid.

The multipotent cells are incorporated (e.g., transplanted) 140 into muscle tissue. The transplantation 140 may be focused on one or more specific areas of muscle tissue. In other embodiments, the transplantation 140 may be more generally intended to apply throughout a patient's body.

In some embodiments, the multipotent cells are injected directly into muscle tissue to be treated.

In some embodiments, the multipotent cells are injected intravenously.

In some embodiments, the treatment method includes a combination of intravenous injection(s) and direct muscle tissue injection(s).

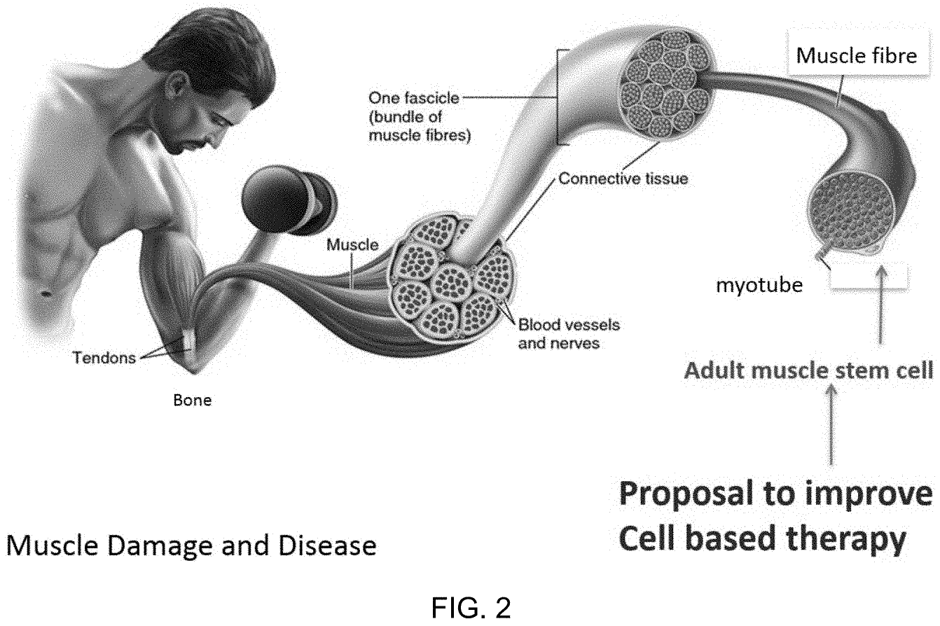

FIG. 2 schematically illustrates a muscle. Each muscle includes a plurality of fascicles. Each fascicle includes a plurality of muscle fibers. Each muscle fiber includes a plurality of myotubes.

In some embodiments, the treatments of the present disclosure are used to treat volumetric muscle loss.

Non-limiting examples of applications for which the MyoD mutants include:
treatment of muscle wasting by improving survival of stem cells or modified stem cells;
treatment of muscle injury by improving survival of stem cells or modified stem cells; and
treatment of muscle dystrophies by improving survival of stem cells, modified stem cells or stem cells modified to treat a specific dystrophy such as DMD.

No other solution targets the molecule (MyoD) responsible for controlling muscle stem cell differentiation as well as controlling/sensitizing myoblasts to apoptotic stimuli.

The following example is merely illustrative and are not intended to limit the disclosure to the materials, conditions, or process parameters set forth therein.

### Examples

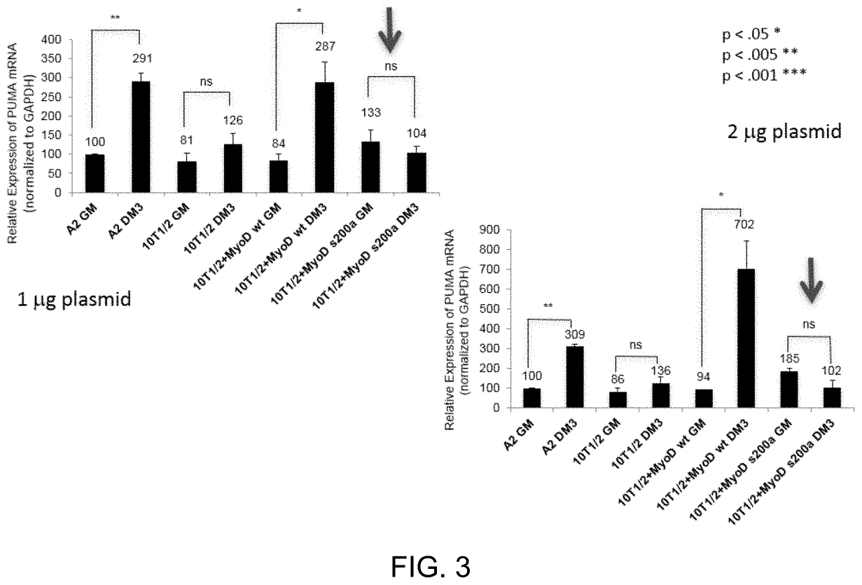

MyoD s200a mutant was transferred into 10T1/2 fibroblasts (stem cells) and the stem cells were tested for PUMA expression and myogenin expression. FIG. 3 includes graphs for PUMA expression after transfection with 1 μg plasmid and 2 μg plasmid. As shown, the MyoD s200a mutant does not support increased PUMA expression (cell death).

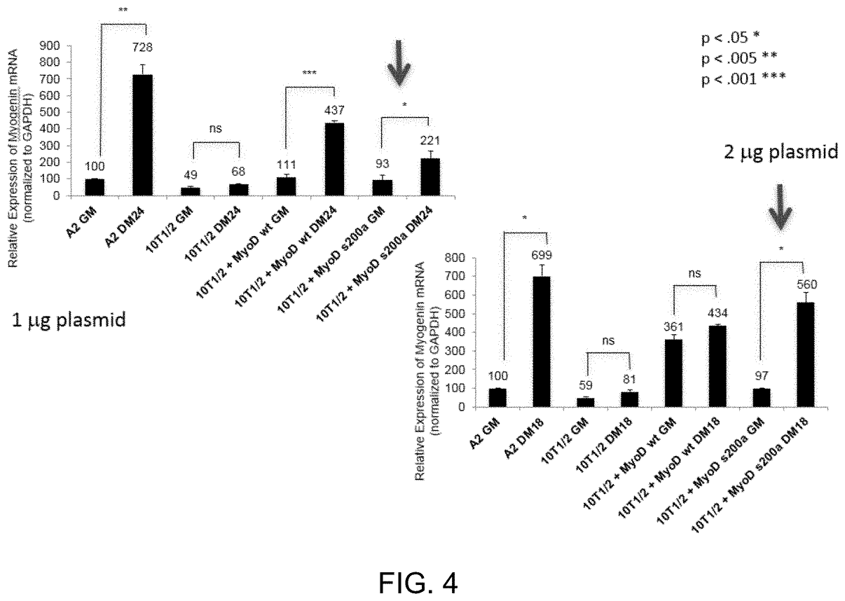

FIG. 4 includes graphs for myogenin expression after transfection with 1 μg plasmid and 2 μg plasmid. As shown, the MyoD s200a mutant supports increased myogenin expression (differentiation).

To determine the impact on proliferation of ectopically expressed MyoD and genetically engineered versions of MyoD, multi-potential fibroblasts were transfected and cells were counted each day over the course of 4 days.

It was determined that MyoD s200a, as well as a genetically engineered version of MyoD with all serines and threonines located next to proline mutated to alanine (7T/S-A), like wild-type MyoD, do not affect the growth rate. However, a genetically engineered version of MyoD with tyrosine mutated to phenylalanine (Y30F) appears to slow the growth rate, possibly making this version less desirable as a therapeutic.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A method for treating a patient with a muscle condition comprises:
transferring multipotent cells into muscle tissue;
wherein a MyoD mutant has been transferred into the multipotent cells.

2. The method of claim 1, wherein the MyoD mutant has been modified to decrease PUMA expression relative to myogenin expression.

3. The method of claim 1, wherein the MyoD mutant is MyoD s200a.

4. The method of claim 1, wherein the MyoD mutant has been modified to replace from 1 to about 8 amino acids.

5. The method of claim 1, wherein the MyoD mutant has been modified to replace at least one instance of serine with alanine.

6. The method of claim 1, wherein the multipotent cells are transplanted via local injection into the muscle tissue.

7. The method of claim 1, wherein the muscle condition is muscular dystrophy.

8. The method of claim 1, wherein the muscle condition is Duchenne muscular dystrophy.

9. The method of claim 1, wherein the muscle condition is an acute muscle injury.

10. The method of claim 1, wherein the muscle condition is muscle wasting.

11. The method of claim 1, wherein the muscle condition is sarcopenia.

12. The method of claim 1, wherein the MyoD mutant is characterized by all serines and threonines located next to proline having been mutated to alanine.

13. A method for treating a patient with a muscle condition comprises:
transferring a MyoD mutant into multipotent stem cells; and
transplanting the multipotent cells into muscle tissue.

14. The method of claim 13, wherein the MyoD mutant is MyoD s200a.

15. The method of claim 13, wherein the MyoD mutant has been modified to replace from 1 to about 8 amino acids.

16. The method of claim 13, wherein the MyoD mutant has been modified to replace at least one instance of serine with alanine.

17. The method of claim 13, wherein the multipotnent cells are transplanted via local injection into the muscle tissue.

18. The method of claim 13, wherein the muscle condition is selected from the group consisting of muscular dystrophy, muscle wasting, and acute muscle injury.

19. The method of claim 13, wherein the transfection is performed using a plasmid.

20. The method of claim 19, wherein the plasmid is a pCS2 plasmid.
